(19) United States
(12) Patent Application Publication
(10) Pub. No.: US 2019/0057504 A1
(43) Pub. Date: Feb. 21, 2019

(54) Image Processor, Image Processing Method, And Image Processing Program

(71) Applicant: KONICA MINOLTA INC., Chiyoda-ku (JP)

(72) Inventor: Tsuyoshi KOBAYASHI, Hachioji-shi (JP)

(21) Appl. No.: 16/105,053

(22) Filed: Aug. 20, 2018

(30) Foreign Application Priority Data
Aug. 18, 2017 (JP) 2017-158124

(51) Int. Cl. G06T 7/00; G06K 9/62; A61B 6/00; A61B 8/08

(52) U.S. Cl. CPC G06T 7/0014; G06K 9/6277; A61B 6/50; A61B 6/5217; G06T 2207/20084; G06T 2207/20081; G06T 2207/30096; G06T 2207/20021; G06T 2207/10116; A61B 8/5223

(57) ABSTRACT

An image processor diagnosing a medical image of a diagnostic target region of a subject imaged by a medical image capturer, includes: an image acquirer acquiring the medical image; and a diagnoser analyzing the medical image using a classifier that has already finished learning and calculating an index indicating a probability of the medical image corresponding to any of categories of lesion patterns, wherein in the classifier, to perform the learning process, in a learning process using the medical image that has been diagnosed not to correspond to any of the categories of lesion patterns, a first value indicating a normal state is set as a correct value of the index, and in a learning process using the medical image that has been diagnosed to correspond to any of the categories of lesion patterns, a second value indicating an abnormal state is set as a correct value of the index.

The entire disclosure of Japanese patent Application No. 2017-158124, filed on Aug. 18, 2017, is incorporated herein by reference in its entirety.

### BACKGROUND

### Technological Field

The present disclosure relates to an image processor, an image processing method, and an image processing program.

### Description of the Related art

Computer-aided diagnosis (hereinafter also referred to as “CAD”), which supports diagnosis of a medical doctor or the like by causing a computer to perform image analysis on a medical image obtained by imaging a diagnostic target region of a subject and presenting an abnormal area in the medical image, is known.

The CAD usually diagnoses whether a particular lesion pattern (for example, tuberculosis or nodule) has appeared in the medical image. For example, the prior art according to the specification of U.S. Pat. No. 5,740,268 discloses a technique of judging whether a pattern of abnormal shadow of a nodule exists in a chest simple X-ray image.

Incidentally, unlike special diagnosis such as tuberculosis screening and extraction of a particular disease in a general practice, in a medical examination, a medical image (for example, a chest simple X-ray image or an ultrasound diagnostic image) is used for viewing by a medical doctor or the like and whether this medical image does not correspond to any of a plurality of categories of lesion patterns (for example, tuberculosis, nodule, blood vessel abnormality, and the like) is comprehensively diagnosed. Then, when it is diagnosed in the medical examination that the medical image corresponds to some lesion pattern, the medical image is sent to a thorough examination.

In this sort of medical examination, there are many lesion patterns that are required to be found from medical images and, for example, there are over 80 types of lesion patterns that are required to be found from chest simple X-ray images or the like. Additionally, in the medical examination, it is required to exhaustively and promptly detect whether the medical image corresponds to any of various lesion patterns.

In this regard, the prior art in the specification of U.S. Pat. No. 5,740,268 and the like have difficulties in detecting a lesion pattern other than a particular lesion pattern such as tuberculosis diagnosis and are not appropriate to use in the above-described medical examination. In other words, since the prior art in the specification of U.S. Pat. No. 5,740,268 and the like have difficulties in judging an abnormal state with respect to a lesion pattern other than a particular lesion pattern, it is difficult to support consultation by a medical doctor who comprehensively diagnoses a health condition.

### SUMMARY

The present disclosure has been made in view of the above disadvantages and it is an object of an aspect of the present invention to provide an image processor, an image processing method, and an image processing program which are more suitable for performing comprehensive diagnosis of a medical image as in the above-described medical examination.

To achieve the abovementioned object, according to an aspect of the present invention, there is provided an image processor that diagnoses a medical image relating to a diagnostic target region of a subject imaged by a medical image capturer, and the image processor reflecting one aspect of the present invention comprises:

an image acquirer that acquires the medical image; and

a diagnoser that performs image analysis on the medical image using a classifier that has already finished learning and calculates an index indicating a probability of the medical image corresponding to any of a plurality of categories of lesion patterns, wherein

in the classifier, in the case of a learning process using the medical image that has been diagnosed not to correspond to any of the plurality of categories of lesion patterns, a first value indicating a normal state is set as a correct value of the index to perform the learning process, and

in the case of a learning process using the medical image that has been diagnosed to correspond to any of the plurality of categories of lesion patterns, a second value indicating an abnormal state is set as a correct value of the index to perform the learning process.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating an example of the overall configuration of an image processor according to an embodiment;

FIG. 2 is a diagram illustrating an example of a hardware configuration of the image processor according to an embodiment;

FIG. 3 is a diagram illustrating an example of the configuration of a classifier according to an embodiment;

FIGS. 4A and 4B are diagrams for explaining a learning process of a learner according to an embodiment;

FIGS. 5A to 5H are diagrams illustrating an example of images used in teacher data of abnormal medical images;

FIGS. 6A to 6H are diagrams illustrating an example of images used in teacher data of abnormal medical images;

FIG. 7 is a diagram illustrating an example of a classifier according to a first modification; and

FIG. 8 is a diagram illustrating an example of a classifier according to a second modification.

### DETAILED DESCRIPTION OF EMBODIMENTS

Hereinafter, one or more preferred embodiments of the present invention will be described in detail with reference to the drawings. However, the scope of the invention is not limited to the disclosed embodiments. Note that, in the present specification and the drawings, the same reference numerals are given to constituent members having substantially the same functional configurations and redundant explanation will be omitted.

[Overall Configuration of Image Processor]

First, the outline of the configuration of an image processor 100 according to an embodiment will be described.

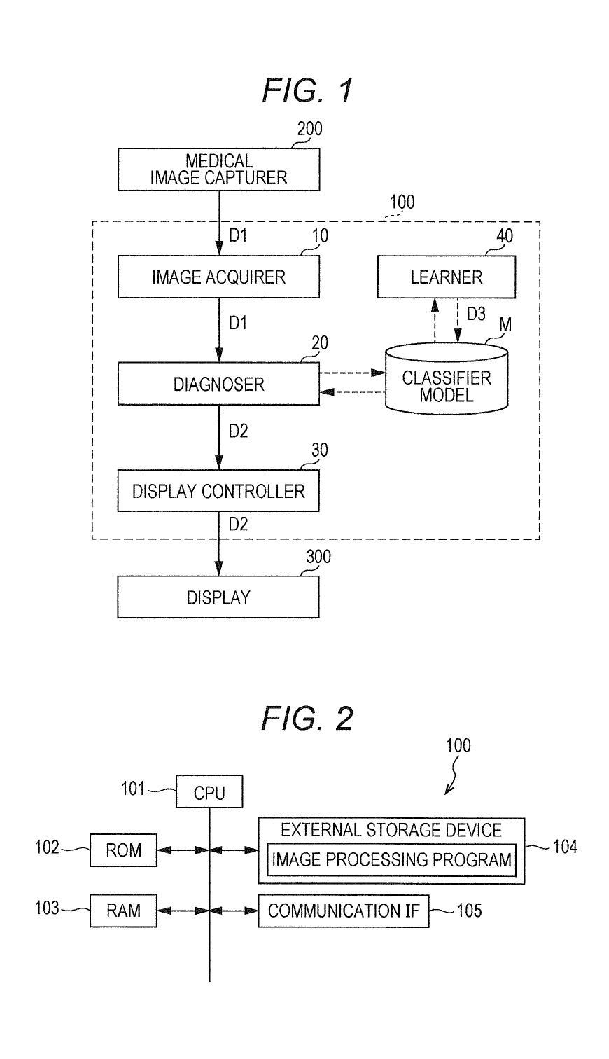

FIG. 1 is a block diagram illustrating an example of the overall configuration of the image processor 100.

The image processor 100 performs image analysis on a medical image generated by a medical image capturer 200 and diagnoses whether this medical image corresponds to any of a plurality of categories of lesion patterns.

The medical image capturer 200 is, for example, a publicly known X-ray diagnostic apparatus. For example, the medical image capturer 200 irradiates a subject with an X-ray and detects an X-ray that has passed through the subject or is scattered by the subject with an X-ray detector, thereby generating a medical image in which a diagnostic target region of the subject is imaged.

A display 300 is, for example, a liquid crystal display and displays a diagnosis result acquired from the image processor 100 in a distinguishable manner to a medical doctor or the like.

FIG. 2 is a diagram illustrating an example of a hardware configuration of the image processor 100 according to the present embodiment.

The image processor 100 is a computer equipped with, as main components, a central processing unit (CPU) 101, a read only memory (ROM) 102, a random access memory (RAM) 103, an external storage device (for example, a flash memory) 104, a communication interface 105, and the like.

For example, the respective functions of the image processor 100 are implemented by the CPU 101 referring to a control program (for example, an image processing program) and various types of data (for example, medical image data, teacher data, and model data of a classifier) stored in the ROM 102, the RAM 103, the external storage device 104, and the like. In addition, the RAM 103 functions as, for example, a work area and a temporary save area for the data.

However, part or all of these functions may be implemented by the process by a digital signal processor (DSP) instead of or in coordination with the process by the CPU. Likewise, part or all of these functions may be implemented by the process by a dedicated hardware circuit instead of or in coordination with the process by software.

The image processor 100 according to the present embodiment is equipped with, for example, an image acquirer 10, a diagnoser 20, a display controller 30, and a learner 40.

[Image Acquirer]

The image acquirer 10 acquires data D1 of a medical image in which a diagnostic target region of a subject is imaged from the medical image capturer 200.

The image acquirer 10 may be configured to directly acquire the image data D1 from the medical image capturer 200 when acquiring the image data D1, or may be configured to acquire the image data D1 held in the external storage device 104 or the image data D1 provided via an Internet line or the like.

[Diagnoser]

The diagnoser 20 acquires the data D1 of the medical image from the image acquirer 10 to perform image analysis on the medical image using a classifier M that has already finished learning and calculates the probability of the subject corresponding to any of a plurality of categories of lesion patterns.

The diagnoser 20 according to the present embodiment calculates the “degree of normality” as an index indicating the probability of the medical image corresponding to any of a plurality of categories of lesion patterns. For example, the “degree of normality” is represented by the degree of normality 100% when the medical image does not correspond to any of a plurality of categories of lesion patterns and represented by the degree of normality 0% when the medical image corresponds to any of a plurality of categories of lesion patterns.

However, the “degree of normality” is an example of the index indicating the probability of the subject corresponding to any of a plurality of categories of lesion patterns, and another index of an arbitrary mode may be used. For example, the “degree of normality” may be in a mode represented by which level value the subject corresponds to out of several stages of level values, instead of the mode represented by a value of 0% to 100%.

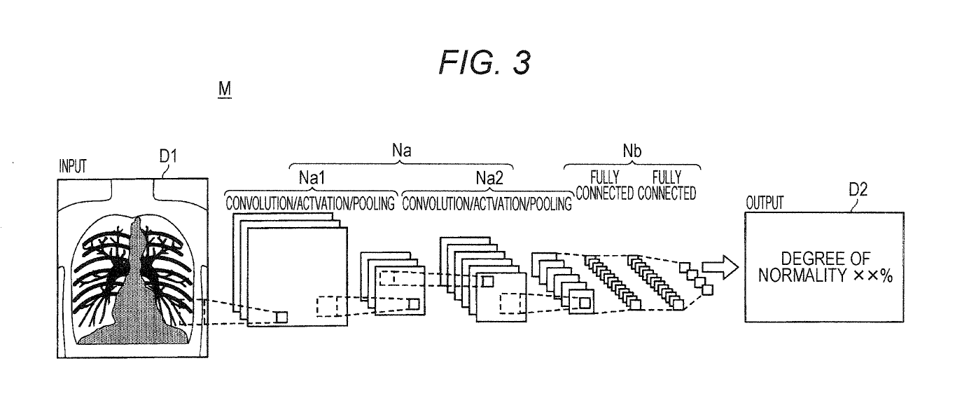

FIG. 3 is a diagram illustrating an example of the configuration of the classifier M according to the present embodiment.

Typically, a convolutional neural network (CNN) is used as the classifier M according to the present embodiment. Note that model data (structure data, learned parameter data, and the like) of the classifier M is held, for example, in the external storage device 104 together with the image processing program.

The CNN has, for example, a feature extractor Na and a classifying member Nb, such that the feature extractor Na carries out a process of extracting image features from an image that has been input and the classifying member Nb outputs a classification result relating to the image in accordance with these image features.

The feature extractor Na is formed by hierarchically linking a plurality of feature amount extraction layers Na1, Na2, . . . . Each of the feature amount extraction layers Na1, Na2, . . . is equipped with a convolution layer, an activation layer, and a pooling layer.

The first layer, namely, the feature amount extraction layer Na1 scans an image that has been input on a predetermined size basis by raster scanning. Then, the feature amount extraction layer Na1 carries out a feature amount extraction process on the scanned data using the convolution layer, the activation layer, and the pooling layer to extract the feature amount included in the input image. The feature amount extraction layer Na1 as the first layer extracts a relatively simple single feature amount such as a linear feature amount extending in a horizontal direction and a linear feature amount extending in an oblique direction.

The second layer, namely, the feature amount extraction layer Na2 scans an image (also referred to as a feature map) input from the feature amount extraction layer Na1 as the previous layer, for example, on a predetermined size basis by raster scanning Then, the feature amount extraction layer Na2 carries out a feature amount extraction process on the scanned data using the convolution layer, the activation layer, and the pooling layer to extract the feature amount included in the input image. The feature amount extraction layer Na2 as the second layer extracts a compound feature amount at a higher class by integrating a plurality of feature amounts extracted by the feature amount extraction layer Na1 as the first layer while taking into consideration the positional relationship therebetween and the like.

The feature amount extraction layers subsequent to the second layer (in FIG. 3, two feature amount extraction layers Na are selectively illustrated for convenience of explanation) execute the same process as the process of the feature amount extraction layer Na2 as the second layer. Then, the output of the feature amount extraction layer as the final layer (each value in the map of the plurality of feature maps) is input to the classifying member Nb.

The classifying member Nb is constituted by a multilayer perceptron in which, for example, a plurality of fully connected layers are hierarchically linked.

The fully connected layer on an input side of the classifying member Nb is fully connected with the respective values in the maps of the plurality of feature maps acquired from the feature extractor Na and the product sum operation is performed on these respective values with different weight coefficients applied to output the resultant values.

The fully connected layer of the classifying member Nb as the next layer is fully connected with values output by respective elements of the fully connected layer as the previous layer and the product sum operation is performed on these respective values with the different weight coefficients applied. Additionally, an output element that outputs the degree of normality is provided at the last stage of the classifying member Nb.

Note that the CNN according to the present embodiment has the same configuration as the publicly known configuration except that a learning process is carried out thereon such that the CNN can output the degree of normality from the medical image.

In general, by performing the learning process beforehand using the teacher data, the classifier M such as the CNN can possess the classification function such that a desired classification result (in this example, the degree of normality) can be output from an image that has been input.

The classifier M according to the present embodiment is configured to employ a medical image as an input (Input in FIG. 3) and output the degree of normality according to the image feature of this medical image D1 (Output in FIG. 3). In addition, the classifier M according to the present embodiment outputs the degree of normality as a value between 0% and 100% depending on the image feature of the input medical image D1.

The diagnoser 20 inputs the medical image to the classifier M that has already finished learning and performs image analysis on this medical image through a forward propagation process by the classifier M to calculate the degree of normality.

Note that a configuration in which the classifier M is capable of receiving inputs of information relating to age, sex, locality, or past medical history in addition to the image data D1 is more suitable (for example, provided as an input element of the classifying member Nb). Features of medical images have correlations with information relating to age, sex, locality, or past medical history. Therefore, a configuration that allows the classifier M to calculate the degree of normality with higher accuracy is enabled by referring to information on age or the like in addition to the image data D1.

In addition to the process by the classifier M, the diagnoser 20 may perform, as a preprocess, a process for converting the size and aspect ratio of the medical image, a color division process for the medical image, a color conversion process for the medical image, a color extraction process, a luminance gradient extraction process, and the like.

[Display Controller]

The display controller 30 outputs data D2 of the degree of normality to the display 300 so as to display the degree of normality on the display 300.

For example, the display 300 according to the present embodiment displays the degree of normality as illustrated in Output in FIG. 3. This numerical value of the degree of normality is used, for example, for judging whether a full-scale examination by a medical doctor or the like is to be performed.

[Learner]

The learner 40 performs a learning process for the classifier M using teacher data D3 such that the classifier M can calculate the degree of normality from the data D1 of the medical image.

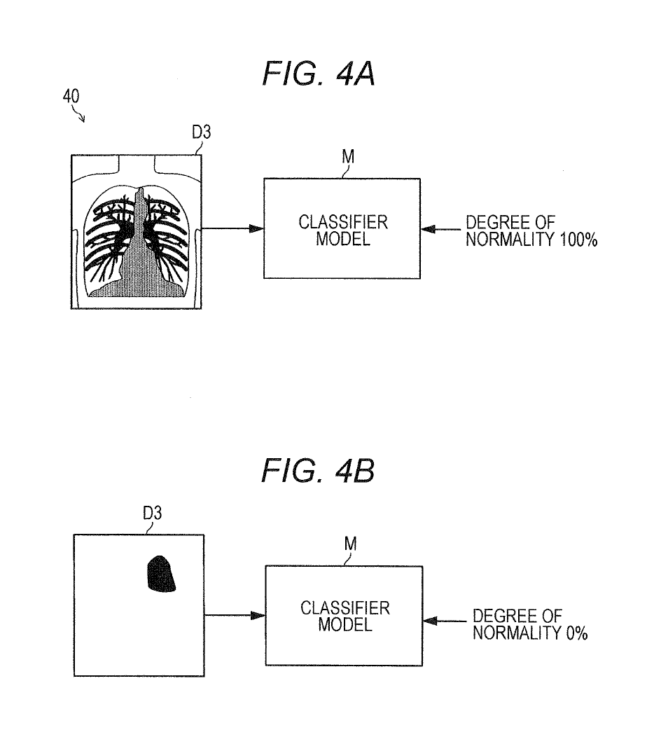

FIGS. 4A and 4B are diagrams for explaining the learning process of the learner 40 according to the present embodiment.

The classification function of the classifier M relies on the teacher data D3 used by the learner 40. The learner 40 according to the present embodiment carries out the learning process as follows, so as to obtain a configuration that allows the classifier M to exhaustively and promptly detect whether the medical image corresponds to one of various lesion patterns.

The learner 40 according to the present embodiment uses, as the teacher data D3, a medical image that has been diagnosed not to correspond to any of the plurality of categories of lesion patterns and a medical image that has been diagnosed to correspond to any of the plurality of categories of lesion patterns, to perform the learning process (hereinafter referred to as “normal medical image teacher data D3” and “abnormal medical image teacher data D3”, respectively). Then, when performing the learning process using the normal medical image teacher data D3, the learner 40 sets a first value indicating a normal state (in this example, the degree of normality 100%) as the correct value of the degree of normality to perform the learning process and, when performing the learning process using the abnormal medical image teacher data D3, sets a second value indicating an abnormal state (in this example, the degree of normality 0%) as the correct value of the degree of normality, to perform the learning process.

In addition, the learner 40 performs the learning process for the classifier M such that, for example, an error (also referred to as loss) of output data with respect to the correct value when an image is input to the classifier M is reduced.

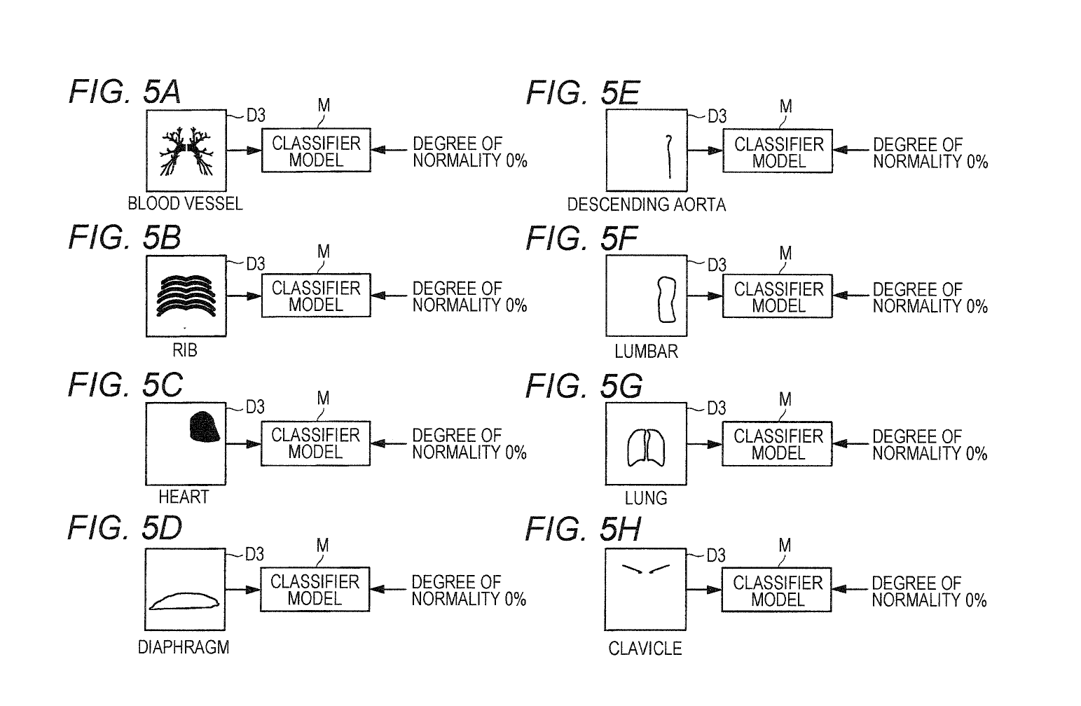

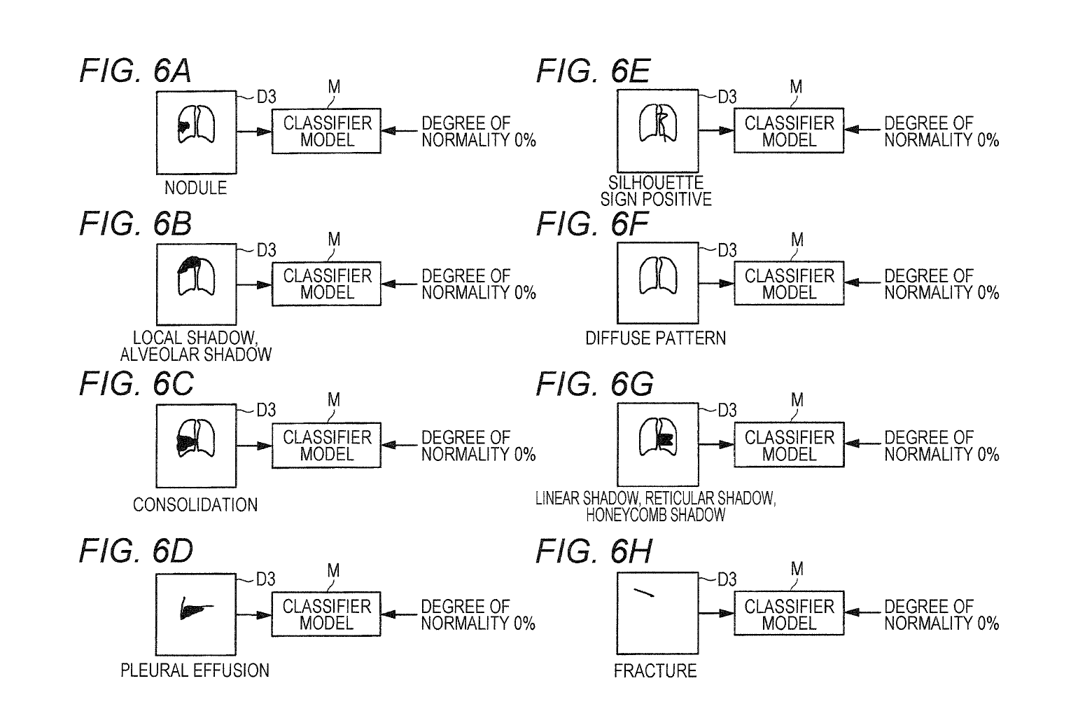

The “plurality of categories of lesion patterns” is reference lesion patterns when a medical doctor or the like judges, from a medical image, that some abnormality has occurred (described later with reference to FIGS. 5A to 5H and 6A to 6H). In other words, the “plurality of categories of lesion patterns” can be any factors usable for judgment as not being in a normal state. There is a plurality of “lesion patterns” required to be found from medical images, including blood vessel contraction as compared with a normal state, presence of unnatural shadow as compared with a normal state, or abnormal shape of the organ as compared with the normal state.

As a consequence of carrying out the learning process in this manner, the classifier M has the classification function of calculating the degree of normality as to whether the medical image corresponds to any of various lesion patterns.

The teacher data D3 of the medical image at this time may be pixel value data or data subjected to a predetermined color conversion process and the like. In addition, data obtained by extracting a texture feature, a shape feature, a spread feature, and the like as a preprocess may be used. Note that the teacher data D3 may be associated with information relating to age, sex, locality, or past medical history in addition to the image data to perform the learning process.

Additionally, the algorithm when the learner 40 performs the learning process can be a publicly known technique. In the case of using the CNN as the classifier M, the learner 40 carries out the learning process on the classifier M using, for example, a publicly known error back propagation method to adjust a network parameter (weight coefficient, bias, and the like). Then, the model data (for example, learned network parameters) of the classifier M on which the learning process has been carried out by the learner 40 is held in the external storage device 104, for example, together with the image processing program.

Furthermore, when performing the learning process using the normal medical image teacher data D3, the learner 40 according to the present embodiment uses the entire image area of the normal medical image to perform the learning process (FIG. 4A). Alternatively, a rectangular area of m×n is selected to learn.

On the other hand, when performing the learning process using the abnormal medical image teacher data D3, the learner 40 according to the present embodiment uses a partial image area obtained by extracting an area of an abnormal state region from the entire image area of the medical image to perform the learning process (FIG. 4B).

As described above, in regard to the abnormal state region, the classifier M selectively uses the image area of this abnormal state region, thereby being enabled to have a higher classification function.

FIGS. 5A to 5H and 6A to 6H are diagrams illustrating an example of images used in the abnormal medical image teacher data D3.

More specifically, FIGS. 5A to 5H are diagrams illustrating image areas of tissues in abnormal states and FIGS. 6A to 6H are diagrams illustrating image areas of shadows in abnormal states.

In more detail, in FIGS. 5A to 5H, a blood vessel area (FIG. 5A), a rib area (FIG. 5B), a heart area (FIG. 5C), a diaphragm area (FIG. 5D), a descending aorta area (FIG. 5E), a lumbar area (FIG. 5F), a lung area (FIG. 5G), and a clavicle area (FIG. 5H) are illustrated as an example of image areas of tissues in abnormal states.

Meanwhile, FIGS. 6A to 6H, a nodule (FIG. 6A), a local shadow and an alveolar shadow (FIG. 6B), consolidation (FIG. 6C), pleural effusion (FIG. 6D), silhouette sign positive (FIG. 6E), a diffuse pattern (FIG. 6F), a linear shadow, a reticular shadow, and a honeycomb shadow (FIG. 6G), and a fracture area (FIG. 6H) are illustrated as an example of image areas of shadows in abnormal states.

The learner 40 performs, for example, a process of cutting out these image areas from the entire image areas, or a binarization process such that these image areas will float out of the entire image areas, to generate the teacher data D3 in which the image areas of abnormal state regions are selectively taken out.

The diagnoser 20 according to the present embodiment performs a diagnostic process on a medical image using the classifier M on which the learning process has been carried out by the above-described technique.

As described above, in the image processor 100 according to the present embodiment, the first value indicating a normal state (in this example, the degree of normality 100%) is set as the degree of normality during the learning process using the medical image not corresponding to any of the plurality of categories of lesion patterns, to perform the learning process on the classifier M. On the other hand, during the learning process using the medical image corresponding to any of the plurality of categories of lesion patterns, the second value indicating an abnormal state (in this example, the degree of normality 0%) is set as the degree of normality to perform the learning process.

Therefore, the image processor 100 according to the present embodiment can exclusively calculate, as a comprehensive degree of normality, whether a medical image corresponds to any of a plurality of categories of lesion patterns. With this configuration, it is possible to mitigate the processing load of image analysis and implement the detection process in a short time while securing the function of exhaustively detecting various lesion patterns.

### First Modification

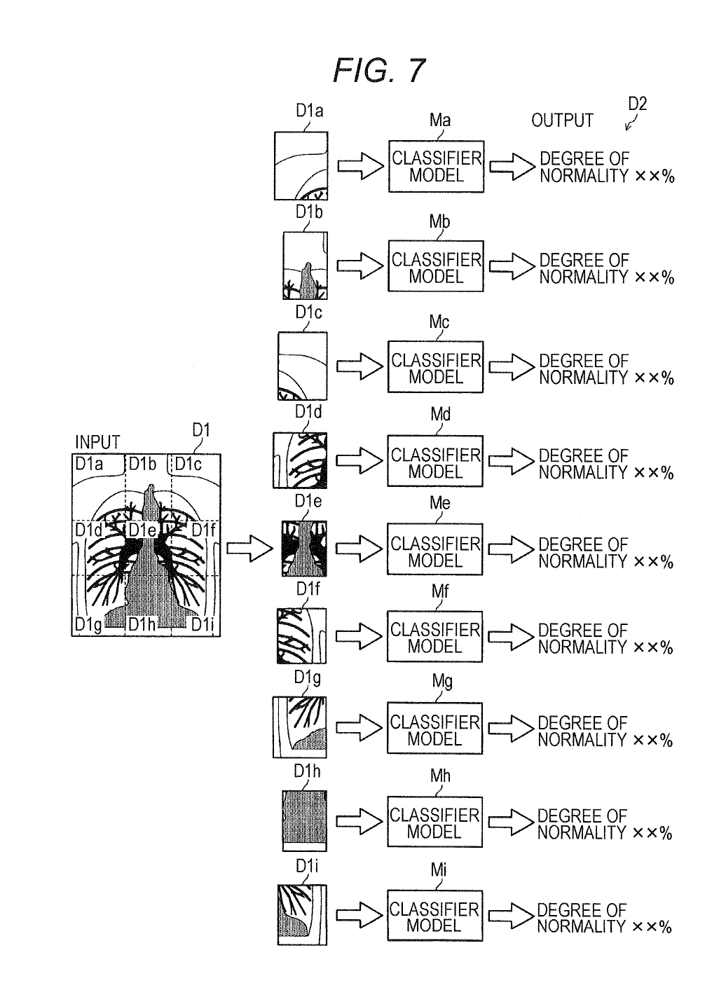

FIG. 7 is a diagram illustrating an example of a classifier M according to a first modification.

A diagnoser 20 according to this first modification differs from that of the above embodiment in dividing the entire image area of the medical image into a plurality of image areas (in this example, dividing into nine areas D1a to D1i) and calculating the degree of normality on the basis of each of these image areas.

The mode according to the first modification can be implemented, for example, by providing the classifier M that performs image analysis on the basis of each image area of the medical image. In FIG. 7, nine different classifiers Ma to Mi are provided so as to correlate with the nine image areas D1a to D1i, respectively. Note that the classifier M that performs image analysis may be provided for each visceral region in the medical image.

For example, a display controller 30 according to this first modification displays the degree of normality calculated on an image area basis on a display 300 in association with the relevant image area of the medical image. For example, the display controller 30 superimposes the degree of normality on the position of an image area of the medical image associated with this degree of normality to display on the display 300.

Meanwhile, the display controller 30 may be configured to display the lowest degree of normality among the respective degrees of normality of the plurality of image areas on the display 300 as the degree of normality of the entire medical image.

Note that the learning process is separately carried out on each of the classifiers Ma to Mi according to the first modification.

### Second Modification

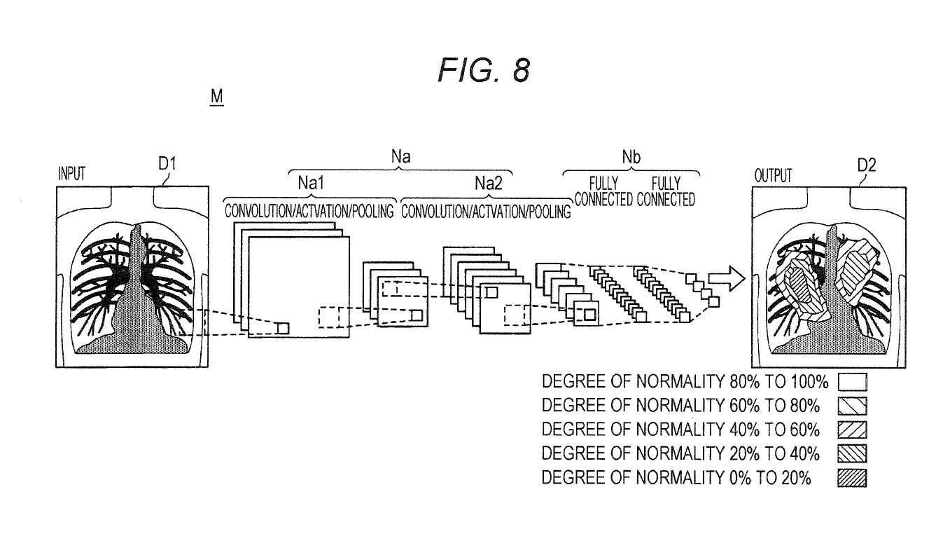

FIG. 8 is a diagram illustrating an example of a classifier M according to a second modification.

A diagnoser 20 according to this second modification differs from that of the above embodiment in calculating the degree of normality on the basis of each pixel area of a medical image (which represents an area of one pixel or an area of a plurality of pixels forming one section; the same applies to the following description).

The mode according to the second modification can be implemented, for example, by providing an output element for each pixel area of the medical image in a classifying member Nb in the CNN (also referred to as regional convolutional neural network (R-CNN)).

For example, a display controller 30 according to this second modification displays the degree of normality of each pixel area on the display 300 in association with the position of the pixel area in the medical image. At this time, for example, the display controller 30 represents the degree of normality of each pixel area by converting the degree of normality into color information and places this color information on top of the medical image to display on the display 300 as a heat map image.

Incidentally, as an example of the heat map image, Output in FIG. 8 illustrates a mode that displays different colors depending on which one of five stages, namely, the degree of normality 0% to 20%, the degree of normality 20% to 40%, the degree of normality 40% to 60%, the degree of normality 60% to 80%, and the degree of normality 80% to 100%, each pixel area corresponds.

By generating the heat map image as in this second modification, for example, it is possible to make it easier for a medical doctor or the like to distinguish an area to be noticed when the medical doctor or the like refers to the medical image.

### Third Modification

An image processor 100 according to a third modification differs from that of the above embodiment in the configuration of a display controller 30.

For example, after calculating the degree of normality of a plurality of medical images, the display controller 30 sets the order of displaying the plurality of medical images on a display 300 based on the degree of normality of each of the plurality of medical images. Then, for example, the display controller 30 outputs the data D1 of the medical images and the data D2 of the degrees of normality to the display 300 in the set order.

Consequently, for example, the plurality of medical images can be displayed on the display 300 in descending order of possibilities of being in an abnormal state such that a subject with higher necessity or urgency can receive a main diagnosis of a medical doctor or the like sooner.

In addition, the display controller 30 may set whether to display each of the plurality of medical images on the display 300, instead of the configuration that sets the order based on the degree of normality of each of the plurality of medical images.

### Other Embodiments

The present invention is not limited to the above embodiments and various modified modes are conceivable.

In the above embodiments, the CNN is indicated as an example of the classifier M. However, the classifier M is not limited to the CNN and any other classifier that can possess the classification function by carrying out the learning process thereon may be used. For example, a support vector machine (SVM) classifier, a Bayes classifier, or the like may be used as the classifier M. Alternatively, a classifier may be configured by a combination of a plurality of these classifiers.

Furthermore, in the above embodiments, examples of the configuration of the image processor 100 are variously indicated. However, it goes without saying that various combinations of the modes indicated in the respective embodiments may be used.

Additionally, in the above embodiments, the X-ray image captured by the X-ray diagnostic apparatus is indicated as an example of the medical image diagnosed by the image processor 100, but the embodiments can be applied to a medical image captured by any other apparatus. For example, the embodiments also can be applied to a medical image captured by a three-dimensional computed tomography (CT) apparatus or a medical image captured by an ultrasound diagnostic apparatus.

Meanwhile, in the above embodiments, the image processor 100 is explained as being implemented by one computer as an example of the configuration thereof, but it is obvious that the image processor 100 may be implemented by a plurality of computers.

In addition, in the above embodiments, the configuration of the image processor 100 equipped with the learner 40 is indicated as an example of the image processor 100. However, if the model data of the classifier M on which the learning process has been carried out is stored in advance in the external storage device 104 or the like, the image processor 100 does not necessarily need to be equipped with the learner 40.

The image processor according to the present disclosure is more suitable for performing comprehensive diagnosis of a medical image.

Although embodiments of the present invention have been described and illustrated in detail, the disclosed embodiments are made for purposes of illustration and example only and not limitation. The scope of the present invention should be interpreted by terms of the appended claims, and the technologies described in the claims include those in which the specific examples exemplified above are modified and changed in a variety of ways.

What is claimed is:

1. An image processor that diagnoses a medical image relating to a diagnostic target region of a subject imaged by a medical image capturer, the image processor comprising:
an image acquirer that acquires the medical image; and
a diagnoser that performs image analysis on the medical image using a classifier that has already finished learning and calculates an index indicating a probability of the medical image corresponding to any of a plurality of categories of lesion patterns, wherein
in the classifier, in the case of a learning process using the medical image that has been diagnosed not to correspond to any of the plurality of categories of lesion patterns, a first value indicating a normal state is set as a correct value of the index to perform the learning process, and
in the case of a learning process using the medical image that has been diagnosed to correspond to any of the plurality of categories of lesion patterns, a second value indicating an abnormal state is set as a correct value of the index to perform the learning process.

2. The image processor according to claim 1, wherein
in the classifier, in the case of the learning process using the medical image that has been diagnosed not to correspond to any of the plurality of categories of lesion patterns, a learning process using an entire image area of the medical image has been performed, and
in the case of the learning process using the medical image that has been diagnosed to correspond to any of the plurality of categories of lesion patterns, a learning process using a partial image area obtained by extracting an abnormal state area from the entire image area of the medical image has been performed.

3. The image processor according to claim 2, wherein
in the classifier, in the case of the learning process using the medical image that has been diagnosed to correspond to any of the plurality of categories of lesion patterns, a learning process using an image area of either a tissue or a shadow in an abnormal state extracted from the entire image area of the medical image has been performed.

4. The image processor according to claim 1, wherein
the diagnoser calculates the index using an entire image area of the medical image as a target.

5. The image processor according to claim 1, wherein
the diagnoser divides an entire image area of the medical image into a plurality of areas and calculates the index on the basis of each of these divided image areas.

6. The image processor according to claim 1, wherein
the diagnoser calculates the index on the basis of each pixel area of the medical image.

7. The image processor according to claim 1, further comprising a display controller that controls a mode of displaying the index on a display.

8. The image processor according to claim 7, wherein
the display controller superimposes the index on a position of an image area of the medical image associated with the index to display the index on the display.

9. The image processor according to claim 8, wherein
the display controller converts the index into color information to display the index on the display.

10. The image processor according to claim 7, wherein
based on the indices calculated for each of a plurality of the medical images, the display controller determines either an order in which the plurality of medical images is displayed on the display or whether to display the plurality of medical images on the display.

11. The image processor according to claim 1, wherein
the medical image is a medical still image.

12. The image processor according to claim 11, wherein
the medical image is a chest simple X-ray image.

13. The image processor according to claim 1, wherein
the classifier comprises a Bayes classifier, a support vector machine (SVM) classifier, or a convolution neural network.

14. The image processor according to claim 1, wherein
the diagnoser calculates the index further based on information relating to age, sex, locality, or past medical history of the subject, in addition to the medical image.

15. An image processing method that diagnoses a medical image relating to a diagnostic target region of a subject imaged by a medical image capturer, the image processing method comprising:
acquiring the medical image; and
performing image analysis on the medical image using a classifier that has already finished learning and calculating an index indicating a probability of the medical image corresponding to any of a plurality of categories of lesion patterns, wherein
in the classifier, in the case of a learning process using the medical image that has been diagnosed not to correspond to any of the plurality of categories of lesion patterns, a first value indicating a normal state is set as a correct value of the index to perform the learning process, and
in the case of a learning process using the medical image that has been diagnosed to correspond to any of the plurality of categories of lesion patterns, a second value indicating an abnormal state is set as a correct value of the index to perform the learning process.

16. A non-transitory recording medium storing a computer readable image processing program causing a computer to execute:
acquiring a medical image relating to a diagnostic target region of a subject imaged by a medical image capturer; and
performing image analysis on the medical image using a classifier that has already finished learning and calculating an index indicating a probability of the medical image corresponding to any of a plurality of categories of lesion patterns, wherein
in the classifier, in the case of a learning process using the medical image that has been diagnosed not to correspond to any of the plurality of categories of lesion patterns, a first value indicating a normal state is set as a correct value of the index to perform the learning process, and
in the case of a learning process using the medical image that has been diagnosed to correspond to any of the plurality of categories of lesion patterns, a second value indicating an abnormal state is set as a correct value of the index to perform the learning process.
